(19) United States
(12) Patent Application Publication
(10) Pub. No.: US 2024/0000417 A1
(43) Pub. Date: Jan. 4, 2024

(54) CRANIAL IMPLANTS WITH INTEGRATED ULTRASOUND-BASED PRESSURE SENSING

(71) Applicants: UNIVERSITY OF UTAH RESEARCH FOUNDATION, Salt Lake City, UT (US); SENTIOMED, INC., Salt Lake City, UT (US)

(72) Inventors: Florian SOLZBACHER, Salt Lake City, UT (US); Navid FARHOUDI, Murray, UT (US); Christopher F. REICHE, Salt Lake City, UT (US); Lars B. LAURENTIUS, Fruitland, UT (US); Thomas ORSINI, Heber City, UT (US)

(21) Appl. No.: 18/343,631

(22) Filed: Jun. 28, 2023

Related U.S. Application Data
(60) Provisional application No. 63/357,294, filed on Jun. 30, 2022.

(51) Int. Cl. A61B 8/08; A61B 8/12

(52) U.S. Cl. CPC A61B 8/0808; A61B 8/12

(57) ABSTRACT

Passive pressure sensors for use in a shunt valve system or other intracranial implants, for measurement of intracranial pressure of cerebrospinal fluid within the brain cavity. Such a pressure sensor implant can be very simple, e.g., including only a micro-membrane configured as a diaphragm, formed of a biocompatible polymer or other material (e.g., PMMA, PDMS, PEEK, polyimide or a biocompatible hydrogel), and an associated air cavity, without any required electronic components. The diaphragm, or air cavity are configured to exhibit changes in response to changes in CSF pressure, which can be detected through ultrasound readout. Ultrasound query at specific frequencies can be used to track such changes which can be correlated to intracranial pressure. Such an ultrasound readout is completely remote, taken from outside the patient's body, without requiring any electronics, wires, etc. for connection to the implant.

### CROSS-REFERENCE TO RELATED APPLICATIONS

This application claims priority to and the benefit of U.S. Provisional Patent Application Ser. No. 63/357,294 entitled CRANIAL IMPLANTS WITH INTEGRATED ULTRASOUND-BASED PRESSURE SENSING filed Jun. 30, 2022 which is herein incorporated by reference in its entirety.

### FIELD OF THE INVENTION

The present invention generally relates to devices and methods for sensing and measurement of intracranial pressure.

### BACKGROUND

Intracranial pressure (ICP) in healthy supine adults is autoregulated. However, in certain medical conditions, the body is unable to regulate such pressure, which can cause brain damage or even death if left untreated. Cranioplasty is one of the oldest surgical procedures, e.g., used for patients with bony voids or defects on the skull caused by brain injuries or operations such as decompressive craniectomy. In most cases, monitoring brain pressure is a valuable tool to avoid severe side effects of increased brain pressure. When this surgery is necessary, ideally, other operations related to insertion and removal of the pressure sensing devices can be avoided by integrating micro-devices inside the cranioplasty implant itself. Hydrocephalus, which is caused by excessive buildup of cerebrospinal fluid (CSF) in the brain ventricles, is one of the conditions that causes abnormal ICP and affects more than 380,000 new individuals annually. Currently, surgical placement of a shunt valve system to redirect the excess CSF is commonly used as a treatment for hydrocephalus patients. These shunts require regular checkups, and the valve needs to be adjusted to avoid under or over drainage of CSF, which imposes a cost and time burden on patients and hospitals.

As such, there is a continuing need for improved systems and methods for monitoring intracranial pressure.

### SUMMARY

An embodiment of the present invention is directed to embedding a miniature completely passive pressure sensor into a hydrocephalus shunt valve system, or placement of such a passive pressure sensor into a cranioplasty implant. Such a pressure sensor can provide data on ICP levels, which could be used to guide adjustment of a shunt valve, or other intervention.

An embodiment according to the present disclosure may include a pressure sensor implant that is very simple, e.g., including only a micro-membrane in the form of a diaphragm formed of a biocompatible material (e.g., biocompatible polymers such as PMMA, PDMS, PEEK, polyimide, or a biocompatible hydrogel). The diaphragm, or a cavity underneath or otherwise near such diaphragm can be patterned or otherwise configured to bend or otherwise exhibit changes in mechanical resonance behavior in response to changes in CSF pressure. Such biocompatible polymer materials are relatively transparent to ultrasound as compared to more rigid metal materials, and off-the-shelf medical ultrasound systems, configured to query at specific ultrasound frequencies can be used to track changes in diaphragm bending or other frequency response or resonance characteristics associated with the diaphragm and/or associated cavity, which changes can be correlated to the desired ICP data. Such an ultrasound readout to determine pressure is completely remote, taken from outside the patient's body, without requiring any electronics, wires, etc. for placement in the body. The employed implanted sensing elements can be completely mechanical, free from electronic components, eliminating any need for subsequent operations related to pressure sensor maintenance or removal. For example, such a passive sensor may simply remain implanted in place, indefinitely, with no removal required. While no electronic components are required, in some embodiments, where desired, electronic components could be provided in conjunction with the pressure sensing implant, e.g., for other purposes, if desired (e.g., for temperature compensation). In an embodiment, no electronic components are implanted.

In an embodiment, ICP can be correlated to bending of the pressure sensing diaphragm within the air or other fluid-filled cavity (filled with air, another gas, or a liquid) included within the implant. Such bending of the diaphragm can manifest itself as a change in how ultrasound waves that interact with such diaphragm and/or cavity are received back and detected at the ultrasound system.

An embodiment is directed to a method for remotely sensing intracranial pressure, the method including providing an implant including a diaphragm formed from a biocompatible material. The diaphragm may cover a cavity (or otherwise be associated with such cavity, as will be explained). The diaphragm is in contact with cerebrospinal fluid (CSF). The diaphragm is configured, e.g., because of its material properties, thickness, etc., to bend based on a pressure difference between the pressure of the CSF and the pressure within the cavity. The diaphragm is queried using an ultrasound transducer of an ultrasound system (e.g., medical ultrasound imaging system), where the ultrasound transducer is configured to emit an ultrasound frequency that is selected to bend or vibrate the diaphragm or selected to be at or near a cavity resonance of the system, which change in CSF pressure is exhibited in changes of bending, vibration or resonance behavior of the diaphragm and/or cavity that exhibits in changes in reflected back “received” ultrasound waves detected by the ultrasound system that includes the ultrasound transducer. Such information on ICP may be used, for example, to adjust a shunt valve or otherwise bring the pressure within the cranial cavity to a desired level.

Another embodiment is directed to a system for remotely sensing intracranial pressure, the system including an implant including a diaphragm formed from a biocompatible material, the diaphragm covering or otherwise being associated with a cavity (e.g., an air-filled cavity), where the diaphragm is in contact with CSF. The diaphragm is configured to bend based on a pressure difference between the pressure of the CSF and the pressure within the cavity. The system further includes, or is configured to be used with an ultrasound system that can be used to query the passive pressure sensor (i.e., the diaphragm and air or other gas-filled cavity). Such an ultrasound system includes an ultrasound transducer configured to emit an ultrasound frequency that is selected to bend and/or cause vibration within the diaphragm or otherwise excite a resonance of the cavity, which change can be detected by the ultrasound system based on the ultrasound waves that are received back at the ultrasound system, after reflection and/or interaction at the pressure sensor.

Another embodiment is directed to an implant device for remotely sensing intracranial pressure. The implant device includes an implant body including a passive pressure sensor in the form of a diaphragm formed from a biocompatible material, the diaphragm covering or otherwise being associated with a cavity, wherein the diaphragm is in contact with cerebrospinal fluid (CSF) during use. The diaphragm is configured to bend based on a pressure difference between the pressure of the CSF and a pressure within the cavity. Such device is configured for use with an ultrasound system including an ultrasound transducer configured to emit an ultrasound frequency that is selected to bend and/or vibrate the diaphragm and/or excite a resonance of the cavity so as to query the passive pressure sensor of the device, wherein the ultrasound system can detect such change based on ultrasound waves that are received back at the ultrasound system, from the pressure sensor.

In an embodiment, ultrasound imaging or intensity data generated by the ultrasound system from query of an interface between the air or other gas-filled cavity and the diaphragm can be correlated to CSF pressure, to determine CSF pressure remotely. While some embodiments may include generation of an ultrasound image (e.g., intensity data from such an image), other embodiments do not require generation or use of any ultrasound image.

In an embodiment, the ultrasound system is used to query the diaphragm and/or cavity at or near a resonance frequency of the diaphragm and/or cavity, and such ultrasound waves are received back at the ultrasound system for detection, which data (from the received ultrasound waves) is used to determine the state of the diaphragm and/or cavity, which can be correlated to CSF pressure.

In an embodiment, a shift in frequency response of the diaphragm can be detected, which shift in frequency response is due to a change in CSF pressure, and such shift in frequency response can be correlated to CSF pressure.

In an embodiment, the dimensions (e.g., diameter or other length/width dimensions, thickness, etc.), as well as the ultrasound frequency used for the query are selected to create a resonance mode in the cavity and/or diaphragm. Such interference pattern can be measured or otherwise determined by tracking intensity of pixels of an ultrasound image corresponding to the location of the cavity, and such intensity data can be correlated to CSF pressure. Depending on query frequency, resonance may occur in the cavity, in the diaphragm, or both. As noted, similar methods can be used to determine CSF pressure, without the need for generation of an ultrasound image (e.g., by simply measuring data relative to frequency response, resonance frequency shift, etc.).

The cavity is filled with a fluid (e.g., a gas) having a significantly lower density than the density of CSF fluid (which the diaphragm is in contact with). In an embodiment, the cavity may simply be filled with air. Other gases could potentially be used (e.g., nitrogen, helium, argon, etc.).

In an embodiment, the diaphragm can be formed from a biocompatible polymer, such as one or more of an acrylic (e.g., polymethylmethacrylate (PMMA)), from polydimethylsiloxane (PDMS), polyimide, polyether ether ketone (PEEK), or from a hydrogel. Other biocompatible polymer materials, or even a thin metal diaphragm configured to bend under ultrasound query, may also be possible. In an embodiment, the diaphragm is advantageously formed from a material that is flexible, rather than rigid, such as a relatively thick metal (e.g., gold). For example, a polymer may be particularly suitable. In another embodiment, the diaphragm could be made of a very thin and sufficiently flexible metal material (e.g., gold foil or the like).

In an embodiment, the system may include a plurality of sensors, each including a corresponding diaphragm covering or otherwise associated with a corresponding cavity, wherein each corresponding diaphragm is in contact with CSF, and wherein the plurality of pressure sensing diaphragms are configured to be sensitive to different pressure ranges (e.g., through differences in thickness, other dimensions, material selection or the like), to allow sensing of different pressure values, depending on CSF pressure. Such pressure sensors may be positioned adjacent to one another (all in the same shunt system or other implant).

In an embodiment, the cavity may be pneumatically separated from the diaphragm so that ultrasound waves used to query the diaphragm do not need to travel through the cavity to reach the diaphragm.

In an embodiment, while the cavity is filled with air or another gas, a liquid filled cavity (e.g., a fluidic microchannel) can also be provided, between the diaphragm and the air or other gas filled cavity, for pneumatically transferring pressure from the diaphragm to the cavity.

In an embodiment, a microfluidic channel and a second diaphragm are provided between the diaphragm and the air or other gas filled cavity for pneumatically transferring pressure from the diaphragm through the microfluidic channel and the second diaphragm to the air or other gas filled cavity. Such a microfluidic channel may be filled with a liquid (e.g., having greater density than the air or other gas-filled cavity). The liquid of the microfluidic channel (e.g., saline) may have a density similar to that of CSF.

In an embodiment, a microfluidic channel is provided between the diaphragm and the air or other gas filled cavity, where bending of the diaphragm during ultrasound query causes displacement of a liquid-gas interface between the microfluidic channel and the air or other gas-filled cavity, which displacement is determined by ultrasound query, and the displacement can be correlated to CSF pressure.

Features from any of the disclosed embodiments may be used in combination with one another, without limitation. This summary is provided to introduce a selection of concepts in a simplified form that are further described below in the detailed description. This summary is not necessarily intended to identify key or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims, or may be learned by the practice of the disclosure as set forth hereinafter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIGS. 1A-1B schematically illustrate how an electrically passive sensor as contemplated herein could be integrated into a hydrocephalus shunt system (FIG. 1A) or integrated into a cranioplasty implant (FIG. 1B). In both schemes, readout can be achieved wirelessly using a standard diagnostic ultrasound system or a stand-alone ultrasound system specially tailored for such purpose.

FIG. 2 schematically illustrates how the displacement of the pressure sensing diaphragm could be detected using ultrasound readout.

FIG. 3 shows results of a finite element analysis (FEA) for bending of a polymer pressure sensing diaphragm due to an applied net pressure from one side (e.g., from an increased CSF pressure).

FIGS. 4A and 4B schematically illustrate exemplary scenarios for moving the air cavity away from the ultrasound query location. In FIG. 4A, the displacement in the diaphragm is transferred pneumatically to the air cavity (through an intermediate liquid cavity). In FIG. 4B displacement in the diaphragm is transferred to another diaphragm, which allows removing the air cavity from between the diaphragm and the ultrasound transducer.

FIG. 5 shows frequency response of a PDMS diaphragm with a diameter of 4 mm and a thickness of 250 μm. The illustrated frequency response shift under different differential pressures is shown when queried at various ultrasound frequencies near 1 MHz.

FIG. 6 schematically illustrates measurement of the bending of a pressure sensing diaphragm by changes in the interference pattern of ultrasound waves reflected in a thin cavity.

FIG. 7 schematically illustrates an exemplary pressure sensing concept based on measurement of the displacement of fluid in a microchannel.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments, and is not necessarily intended to limit the scope of the claimed invention.

The present disclosure is directed to methods and systems for remotely sensing intracranial pressure. An exemplary method includes providing an implant including a diaphragm formed from a biocompatible polymer or other biocompatible material, the diaphragm covering or otherwise being associated with a cavity, wherein the diaphragm is in contact with cerebrospinal fluid (CSF). The diaphragm is configured (e.g., sized and shaped) to bend based on a pressure difference between the pressure of the CSF and a pressure within the cavity of the sensor. The method further includes querying the diaphragm with an ultrasound transducer of an ultrasound system, where the transducer is configured to emit ultrasound waves with a frequency that is specifically selected to bend or vibrate the diaphragm, or is equivalent or close to one of the cavity resonances of the system, which change (bending, vibration or resonance) is detected in the reflected ultrasound waves received and detected by the ultrasound system.

An associated system includes an implant including a passive pressure sensor in the form of a diaphragm formed from a biocompatible polymer or other biocompatible material, incorporated into a cranioplasty implant or hydrocephalus shunt system. The diaphragm covers or otherwise is associated with a cavity, where the diaphragm is in contact (either direct contact or pneumatic contact) with CSF. The diaphragm is configured to bend based on a pressure difference between the pressure of the CSF and a pressure within the cavity of the sensor. The system further includes, or is used with an ultrasound system that is used to query the passive pressure sensor, where the ultrasound system includes an ultrasound transducer configured to emit an ultrasound wave with a frequency that is substantially equal to a cavity resonance frequency or is selected to bend and/or vibrate the diaphragm, which results in bending or vibration of the diaphragm (e.g., or creation of a resonance mode in the cavity and/or diaphragm). The ultrasound system is configured to detect such changes based on ultrasound waves that are reflected and received back at the ultrasound system, from the pressure sensor. Such received data is correlated to the CSF pressure within the patient.

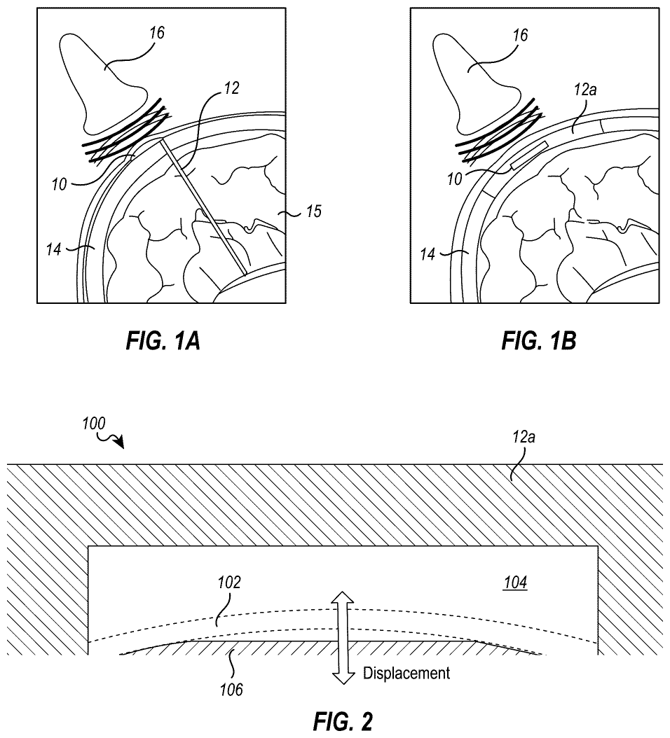

FIGS. 1A-1B generally illustrate such a concept. For example, FIG. 1A schematically illustrates integration of a pressure sensing element 10 within a hydrocephalus shunt system 12. Such a sensor may be provided, in communication with the shunt, implanted within the cranium/skull 14. FIG. 1A illustrates how a simple ultrasound transducer 16 can be used for the read-out of the sensor (pressure sensing element) 10. FIG. 1B illustrates a similar configuration, showing integration of a pressure sensing element 10 within a cranioplasty implant 12a. Such systems allow monitoring of the pressure applied to the brain 15.

A variety of general sensing modalities could be used in conjunction with ultrasound readout, to detect changes in the bending of the pressure sensing diaphragm within the implant. Advantageously, with any of the described embodiments, ultrasound readout may be achieved using commercially available ultrasound, use of a non-destructive testing module, or a custom-built solution. Of course, an advantage of using commercially available ultrasound imaging systems is the overall simplicity and low cost, as a facility where such monitoring may occur will most likely already have access to such an ultrasound system.

The cavity associated with the presently described pressure sensor embodiments may be fully sealed or may be configured to be accessible to allow for recalibration or other maintenance through a capillary from the outside of the implant.

FIG. 2 shows a simple configuration of an implant 100, where ultrasound may be used to determine bending of the pressure sensor diaphragm 102. For example, using conventional commercially available ultrasound systems and query frequencies, it may generally be possible to measure or detect a bending change on the order of about 0.5 mm. While there may not be sufficient resolution to directly measure bending, associated changes in the reflected ultrasound waves can be detected and measured, to determine CSF pressure. For example, changes due to vibration, bending, or resonance can be measured or otherwise determined by tracking intensity of pixels of an ultrasound image corresponding to the location of the cavity, such intensity data being correlated to CSF pressure. As shown in FIG. 2, the implant 100 may include a sensing structure including a diaphragm 102 that covers a small cavity 104 within the bulk of implant 100. Diaphragm 102 is in contact with the CSF 106, and is configured to bend based on any pressure difference between the CSF on one side of the diaphragm, and the absolute pressure within cavity 104 on the other side of such diaphragm 102. For example, where CSF pressure is greater than the pressure within cavity 104, diaphragm 102 will bend upwardly, as shown, due such pressure differential.

Because there is an abrupt and significant difference in acoustic impedance between the cavity 104 (e.g., filled with air or another gas) and the diaphragm 102, this interface has high contrast, and is readily visible in an ultrasound image of such a region. The ultrasound images (or other associated data) obtained while bending occurs on this interface can be correlated to CSF pressure, using simple calibration.

The size and thickness of the diaphragm 102 may be selected based on the contemplated operating pressure range and the material properties of the diaphragm. Finite element modeling analytical calculations, and/or other methods may be used to guide design for a proper range of displacement to be detected within the limits of ultrasound resolution and the contemplated pressure range. For example, a typical “normal” intracranial gauge pressure within normal human adults may range from about 5 mm Hg to about 15 mm Hg. Exemplary elevated intracranial pressures may be greater than 20 mm Hg, or greater than 25 mm Hg (e.g., up to 100 mm Hg, up to 70 mm Hg, or up to 50 mm Hg).

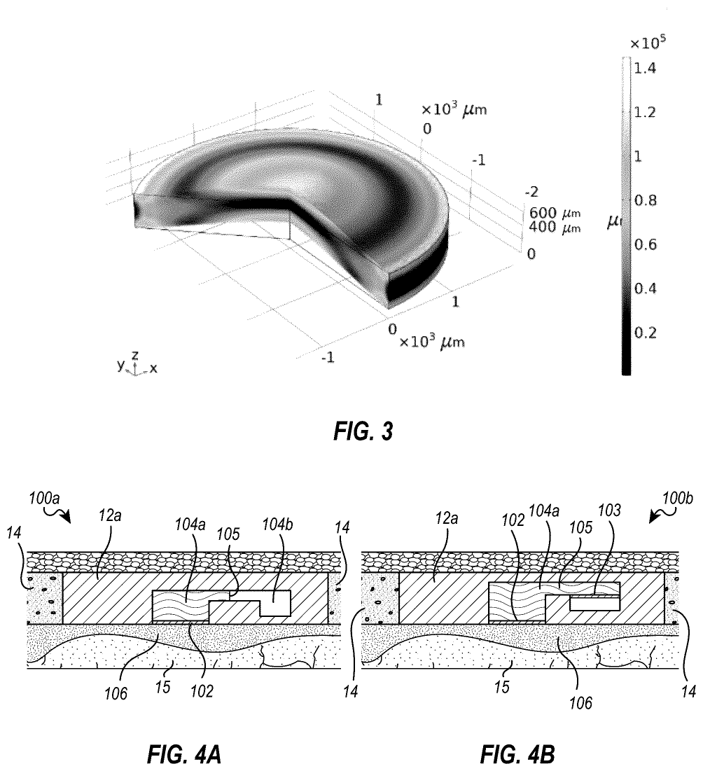

FIG. 3 shows an exemplary finite element modeling of a polymer pressure sensing diaphragm bending due to an applied net pressure from one side. The image shown is for an example of a finite element simulation for a PDMS diaphragm with a diameter of 4 mm, and a 500 μm thickness. Another exemplary diaphragm may be a polyimide diaphragm with a 5 mm diameter, a thickness of 25 μm. Cavity thickness (e.g., height) may be 126 μm. In an embodiment, a plurality of diaphragms/pressure sensors may be provided, each configured to detect a different pressure range. For example, such a plurality of sensors could be positioned side by side, and imaged with an ultrasound transducer array positioned remotely (outside the patient's head), to provide readout over a desired range of pressure range values. For example, one pressure sensor may be particularly configured to detect pressures within a “normal” range of 5-20 mm Hg, another pressure sensor may be configured to detect moderately elevated pressures within a range of 20 mm Hg to 50 mm Hg. Another pressure sensor may be configured to detect higher elevated pressures within a range of 50 mm Hg to 100 mm Hg. The diaphragms of such pressure sensors may differ from one another in their thickness, diameter, or other characteristics.

In an embodiment, an exemplary diaphragm may have a thickness greater than 5 μm, greater than 10 μm, greater than 20 μm, greater than 30 μm, greater than 40 μm, greater than 50 μm, greater than 70 μm, greater than 80 μm, greater than 90 μm, greater than 100 μm, greater than 150 μm, greater than 200 μm, less than 1000 μm, less than 750 μm, less than 600 μm, less than 500 μm, less than 400 μm, or less than 300 μm. By way of further example, an exemplary diaphragm may have a diameter of at least 0.05 mm, at least mm, at least 0.5 mm, less than 20 mm, less than 10 mm, or less than 5 mm. For example, an exemplary diaphragm has a thickness of 250 μm or 500 μm and a diameter of 4 mm, or a thickness of 25 μm and a diameter of 5 mm. Such values are of course merely provided as examples.

In some instances, there may be a significant attenuation of ultrasound waves caused by the air cavity, in such a manner that the ultrasound waves are blocked from reaching the pressure sensing diaphragm. Such may be particularly problematic where the air cavity is longitudinally axially in line with the ultrasound transducer and the diaphragm (i.e., all are in the same axis, with the air cavity in between the ultrasound transducer and the diaphragm). In order to address such an issue, the air cavity can be moved away from the area between the ultrasound probe of the ultrasound system and the pressure sensing diaphragm. FIGS. 4A and 4B illustrate two non-limiting ways to accomplish such. For example, as shown in implant 100a of FIG. 4A, a liquid filled cavity portion 104a is provided, which transfers the pressure to an air cavity portion 104b on a lateral side, using a microchannel 105 extending between cavity portions 104a and 104b. Such an embodiment includes a liquid in cavity portion 104a in pneumatic contact with the pressure sensing diaphragm 102. Such contact may aid in more efficiently transferring ultrasound waves from the ultrasound transducer probe to the diaphragm 102.

Another configuration is shown in FIG. 4B, where the implant 100b is similarly configured, but pressure from CSF 106 is transferred from the first diaphragm 102 (located at the interface between the CSF 106 and liquid filled cavity portion 104a) to a second diaphragm 103 using a microfluidic channel 105. In such a configuration, the air cavity portion 104b is shown as positioned under the 2n d diaphragm 103, so that the ultrasound waves do not have to travel through the air cavity 104b in order to reach diaphragm 102.

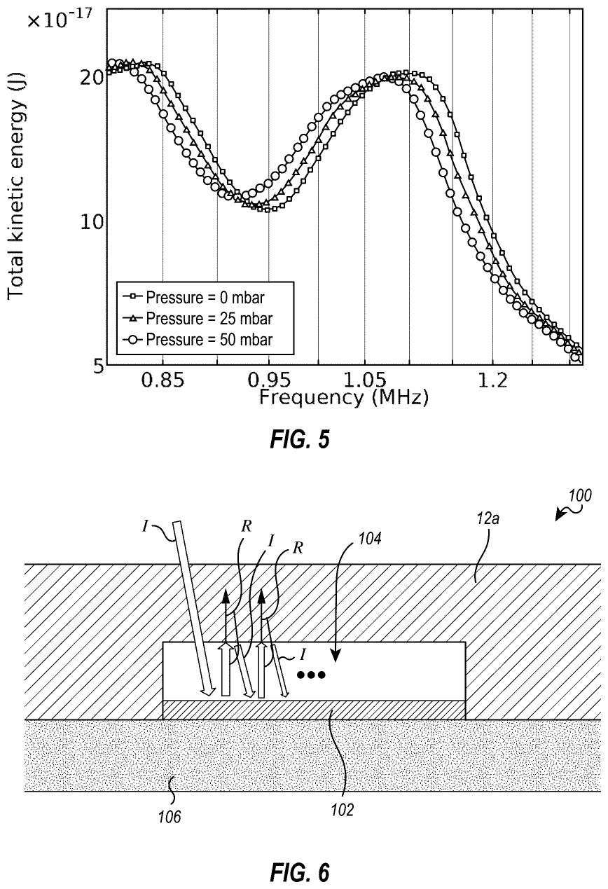

Another measurement modality may involve measuring the bending of a diaphragm using the changes in resonance response due to applied stress and/or strain. In such an embodiment, the sensing structure may similarly include a diaphragm that covers a small cavity in the bulk of the implant. The diaphragm is in contact with the CSF and can bend based on the pressure difference between the CSF and the absolute pressure within the cavity (e.g., cavity filled with air or other gas). In different bending states, the diaphragm experiences lateral tensions that cause a change in the mechanical frequency response of the diaphragm. When pressure is applied to the diaphragm, the bending on the diaphragm causes an outward tension alongside the edges of the diaphragm. This tension causes a change in resonance frequency of the diaphragm, as shown in FIG. 5, similar to tuning a guitar by changing the tension on its strings. The diaphragm can be excited (queried) with ultrasound waves, thus absorbing mechanical energy from them when the querying ultrasound wave is at or near the resonance frequency of the diaphragm. The intensity of the reflected waves can be monitored using the ultrasound transducer probe to obtain information about the resonance spectrum, and thus the bending state of the diaphragm, and thus the ICP. In the case of ultrasound imaging, the intensity of the pixels corresponding to the spatial location of the diaphragm can be tracked to obtain information about the bending state of the diaphragm. While monitoring the intensity of the pixels (and thus using a generated ultrasound image) may be used in such analysis, it will be appreciated that the contemplated methods do not necessarily require generation of an ultrasound image. For example, shift in the frequency response of the reflected waves can simply be used to calculate ICP, without the need to generate an ultrasound image as a necessary intermediate. Similar methodology applies with cavity resonance and smaller cavity and pressure and thus speed of sound changes in the cavity.

Referring again to FIG. 5, there is shown a frequency response of a polydimethylsiloxane (PDMS) “silicone” diaphragm with a diameter of 4 mm, and a thickness of 250 μm, queried at ultrasound frequencies near 1 MHz. FIG. 5 shows the change in frequency response at different applied pressure differentials, e.g., with no applied pressure differential, with an applied differential of 25 mbar (18.75 mm Hg), and an applied differential of 50 mbar (37.5 mm Hg). As shown, absorption peaks shift, depending on applied pressure differential. Such a detected shift in frequency response (e.g., frequency absorption) may be correlated with ICP, as will be apparent. No ultrasound image need be generated to make such a determination.

FIG. 6 illustrates how determination of ICP may be based on measurement of diaphragm bending using ultrasound interference or cavity resonance inside the cavity of the sensor. For example, as shown, the sensing structure may include a diaphragm 102 that covers a small cavity 104 in the bulk of the implant 100, as described previously. The diaphragm 102 is in contact with the CSF 106 and can bend based on the pressure difference between the CSF and the absolute pressure within cavity 104. The sharp transition in acoustic impedance at the interface of the cavity 104 with the bulk surrounding implant 100, as well as the similar mismatch in acoustic impedance between the cavity 104 and the diaphragm 102, can result in multiple ultrasound reflections within cavity 104 during ultrasound query. In FIG. 6, the incoming waves are labeled “I”, while the reflected waves are labeled “R”. Given a correct combination of ultrasound frequency used for the query, and proper selection of cavity dimensions, these reflections can interfere with one another in a constructive or destructive manner, depending on their relative phase angles, e.g., in a similar fashion as occurs in Fabry-Perot interferometers used in optics. Slight changes in the thickness of the cavity 104 can cause abrupt changes in the interference pattern, which can be measured by tracking the intensity of the reflected waves. In medical ultrasound imaging, tracking intensity of pixels corresponding to the spatial location of the cavity 104 can give information about the bending state of the diaphragm. Of course, other methods, that do not rely on generation of an ultrasound image can also be used.

By way of example, the air or other gas-filled cavity as described herein may have a length or diameter that is sized similarly to the diaphragm, e.g., at least 0.05 mm, at least 0.1 mm, at least 0.5 mm, less than 20 mm, less than 10 mm, or less than 5 mm. The height of the air cavity may be at least 10 μm, or at least 25 μm, such as from 10-500 μm, or 10-240 μm. Exemplary values, also useful as lower or upper endpoints of any range, include 50 μm, 70 μm, 100 μm, 125 μm (or 126 μm), 140 μm, 150 μm, 200 μm, 240 μm, 280 μm, 300 μm, 350 μm, 400 μm, 450 μm or 500 μm.

Cavity height and depth, and pressure within the cavity are also important. In an embodiment, cavity pressure may be within 20%, 10%, or within 5% of typical room (i.e., atmospheric) pressure, and or within such ranges relative to the typical or maximum expected diagnostic ICP pressures as noted herein.

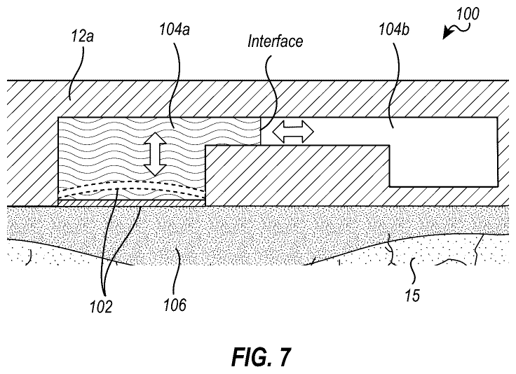

FIG. 7 illustrates monitoring of pressure by measuring fluid displacement in a microchannel, which is another modality that may be used to determine ICP using an implant with a diaphragm sensor as described herein. As shown in FIG. 7, the sensing structure includes a diaphragm 102 that covers a small cavity in the bulk of implant 100. The diaphragm 102 is in contact with the CSF 106, and can bend based on the pressure difference between the CSF 106 and the absolute pressure of the cavity. The cavity is shown as including a liquid filled portion 104a and an air filled portion 104b, connected through a microchannel 105. The air cavity portion 104b is pneumatically connected to the diaphragm through microchannel 105, as shown. The location of the fluid-air interface in the microchannel 105 changes as the diaphragm 102 bends, and this location can be measured (e.g., using ultrasound imaging or otherwise), and correlated to CSF pressure.

Because the acoustic impedance difference between the liquid filled cavity portion 104a and the bulk of the implant 100 is far smaller than the acoustic impedance difference between the air filled cavity portion 104b and the bulk of the implant 100, the location of the liquid-gas interface inside the microchannel 105 will show up in an ultrasound image with high contrast, making measurement easier. Markers may be included on any of the described embodiments, to facilitate easy location and identification of the sensor location. The dimensions of the microchannel 105 can be adjusted to maximize the fluid-air interface displacement, increasing the sensing resolution significantly (e.g., a small change in pressure differential equates to a large displacement in the fluid-air interface). Finally, the microchannel can be designed with any of various geometries, allowing squeezing a significant amount of horizontal information under the ultrasound system probe.

In an embodiment, the microfluidic channel is elongate as shown, e.g., with a width of any desired dimension, e.g. 0.01 mm to 2 mm, or 0.05 mm to 1 mm, and a length significantly greater than the width, e.g., of perhaps 0.5 to 10 mm, 0.5 mm to 5 mm, or 0.5 to 3 mm.

Any of the described systems or methods may include a mechanism to compensate for temperature variances in the patient's body and/or external pressure variations (e.g., measurement of such ambient temperature and/or pressure, and calibration and/or compensation for such).

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used throughout this application the words “can” and “may” are used in a permissive sense (i.e., meaning having the potential to), rather than the mandatory sense (i.e., meaning must). Additionally, the terms “including,” “having,” “involving,” “containing,” “characterized by,” as well as variants thereof (e.g., “includes,” “has,” “involves,” “contains,” etc.), and similar terms as used herein, including within the claims, shall be inclusive and/or open-ended, shall have the same meaning as the word “comprising” and variants thereof (e.g., “comprise” and “comprises”), and do not exclude additional un-recited elements or method steps, illustratively. Unless defined otherwise, all technical and scientific terms used herein have the same meaning as commonly understood by one of ordinary skill in the art to which the present disclosure pertains.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition.

Disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure. Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

Accordingly, the present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

What is claimed is:

1. A method for remotely sensing intracranial pressure, the method comprising:
(a) providing an implant including a diaphragm formed from a biocompatible material, the diaphragm covering or otherwise being associated with a cavity, wherein the diaphragm is in contact with cerebrospinal fluid (CSF);
(b) wherein the diaphragm is configured to bend based on a pressure difference between the pressure of the CSF and a pressure within the cavity; and
(c) querying the diaphragm with an ultrasound transducer of an ultrasound system, wherein the ultrasound transducer is configured to emit an ultrasound frequency that is selected to bend or vibrate the diaphragm and/or excite a resonance of the cavity, which change is detected in received ultrasound waves detected by the ultrasound system.

2. The method of claim 1, wherein ultrasound image or intensity data generated by the ultrasound system from query of the cavity or an interface between the cavity and the diaphragm is correlated to CSF pressure, so as to determine CSF pressure.

3. The method of claim 1, wherein the ultrasound system is used to query the diaphragm at or near a resonance frequency of the diaphragm, and received and detected ultrasound waves from such query are used to determine a bending state of the diaphragm, which is correlated to the CSF pressure.

4. The method of claim 3, wherein a shift in frequency response of the diaphragm is detected, which shift in frequency response is correlated to CSF pressure.

5. The method of claim 1, wherein dimensions of the cavity and the ultrasound query frequency are selected to create a resonance mode in the cavity and/or diaphragm, wherein changes associated with such are measured or otherwise determined by tracking intensity of pixels of an ultrasound image corresponding to a location of the cavity, such intensity data being correlated to CSF pressure.

6. A system for remotely sensing intracranial pressure, the system comprising:
(a) an implant including a passive pressure sensor in the form of a diaphragm formed from a biocompatible material, the diaphragm covering or otherwise being associated with a cavity, wherein the diaphragm is in contact with cerebrospinal fluid (CSF);
(b) wherein the diaphragm is configured to bend based on a pressure difference between the pressure of the CSF and a pressure within the cavity; and
(c) wherein the system includes or is associated with an ultrasound system that can be used to query the passive pressure sensor, wherein the ultrasound system includes an ultrasound transducer configured to emit an ultrasound frequency that is selected to bend and/or vibrate the diaphragm and/or excite a resonance of the cavity, wherein the ultrasound system is configured to detect such change based on ultrasound waves that are received back at the ultrasound system, from the pressure sensor.

7. The system of claim 6, wherein the system includes one or more markers for identifying a position of the diaphragm and/or the cavity.

8. The system of claim 6, wherein the cavity is filled with air or another gas.

9. The system of claim 6, wherein the diaphragm is formed from one or more of polymethylmethacrylate (PMMA), polydimethylsiloxane (PDMS) polyether ether ketone (PEEK), polyimide, or a hydrogel.

10. The system of claim 6, wherein dimensions of the cavity and ultrasound query frequency are selected to provide an interference pattern or resonance mode between ultrasound reflections within the cavity when queried, wherein such interference pattern or resonance mode can be measured or otherwise determined by tracking intensity of pixels of an ultrasound image corresponding to a location of the cavity, and such intensity data can be correlated to CSF pressure.

11. The system of claim 6, wherein the implant includes a plurality of pressure sensors, each including a corresponding diaphragm formed from a biocompatible polymer, each corresponding diaphragm covering or otherwise being associated with a corresponding cavity, wherein each corresponding diaphragm is in contact with CSF, wherein the plurality of pressure sensors are configured to be sensitive to different pressure ranges, wherein the plurality of pressure sensors are positioned adjacent one another.

12. The system of claim 6, wherein the cavity is pneumatically separated from the diaphragm so that ultrasound waves used to query the diaphragm do not need to travel through the cavity to reach the diaphragm.

13. The system of claim 6, wherein the system further comprises a mechanism to compensate for temperature variances in a patient's body and/or external pressure variations.

14. The system of claim 12, wherein the cavity is an air or other gas filled cavity, wherein a liquid filled microchannel is provided between the diaphragm and the air or other gas filled cavity for pneumatically transferring pressure from the diaphragm to the air or other gas filled cavity.

15. The system of claim 12, wherein the cavity is an air or other gas filled cavity, wherein a microfluidic channel and a second diaphragm are provided between the diaphragm and the air or other gas filled cavity for pneumatically transferring pressure from the diaphragm through the microfluidic channel and the second diaphragm to the air or other gas filled cavity.

16. The system of claim 15, wherein the microfluidic channel is filled with liquid.

17. The system of claim 6, wherein dimensions of the cavity and ultrasound query frequency are selected to provide an interference pattern or resonance mode between ultrasound reflections within the cavity when queried, wherein such interference pattern or resonance mode can be measured or otherwise determined by tracking intensity of pixels of an ultrasound image corresponding to a location of the cavity, and such intensity data can be correlated to CSF pressure.

18. The system of claim 6, wherein the cavity is an air or other gas filled cavity, wherein a microfluidic channel is provided between the diaphragm and the air or other gas filled cavity, wherein bending or vibration of the diaphragm during ultrasound query causes displacement of a liquid-gas interface between the microfluidic channel and the air or other gas filled cavity, which displacement is determined by ultrasound query, the displacement being correlated to CSF pressure.

19. An implant device for remotely sensing intracranial pressure comprising:
(a) an implant body including a passive pressure sensor in the form of a diaphragm formed from a biocompatible material, the diaphragm covering or otherwise being associated with a cavity, wherein the diaphragm is in contact with cerebrospinal fluid (CSF) during use;
(b) wherein the diaphragm is configured to bend based on a pressure difference between the pressure of the CSF and a pressure within the cavity; and
(c) wherein the device is configured for use with an ultrasound system including an ultrasound transducer configured to emit an ultrasound frequency that is selected to bend and/or vibrate the diaphragm and/or excite a resonance of the cavity so as to query the passive pressure sensor of the device, wherein the ultrasound system can detect such change based on ultrasound waves that are received back at the ultrasound system, from the pressure sensor.
